(19) United States
(12) Patent Application Publication
(10) Pub. No.: US 2006/0083759 A1
(43) Pub. Date: Apr. 20, 2006

(54) Stabilization of the profile of release of active substances from a formulation

(76) Inventors: Aleksander Resman, Ljubljana (SI); Darja Fercej-Temeljotov, Ljubljana (SI); Vlasta Humar, Stahovica (SI); Marko Opresnik, Ljubljana (SI)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/521,295

(22) PCT Filed: Jul. 15, 2002
(86) PCT No.: PCT/SI03/00025

§ 371 Date: May 17, 2005

(30) Foreign Application Priority Data
Jul. 17, 2002 (SI) P-200200179

(51) Int. Cl. A61K 9/00

(52) U.S. Cl. 424/400

(57) ABSTRACT

The invention discloses a method of a physical pre-treatment of an active substance, which modifies technologically important physical properties of the active substance so as to enable the manufacture of a formulation having a more stable release profile of the active substance over the whole shelf life of the medicine than the profile would be with the same composition, but without pre-treatment.

### TECHNICAL FIELD

IPC: A 61 K 9/52 (C 07 G 11/00)

The present invention belongs to the field of pharmaceutical technology and relates to the stabilization of the profile of release from a formulation containing a high dosage active substance that is poorly soluble in an aqueous medium.

More specifically, the present invention relates to a method for a physical pre-treatment of an active substance, by which treatment the technologically important physical properties of the active substance are modified so as to enable the manufacture of a more stable formulation having a stable and reproducible release profile over the whole shelf life of the medicine.

### TECHNICAL PROBLEM

It is well known that several active substances have technologically unfavourable properties and/or the release of the active substance from the dosage form is poor or inadequate.

By comparing the profiles it has been established that the release rate of a high dosage active substance being poorly soluble in an aqueous medium from a formulation changes with aging, which is even more expressed in the conditions of an accelerated stability assay.

The present invention is based on the need to find a simple and effective method of pre-treatment of such an active substance, which will reduce the effect of storage time or aging as much as possible so that over the whole shelf life the release rate will provide for optimum and reproducible blood concentrations of the active substance in order to achieve therapeutical effects over an extended period of time.

### PRIOR ART

By investigating the ways for stabilizing the profile of the release of an active substance from a pharmaceutical formulation, several references, mostly articles, were found. Raghunatan et al. pre-treated a complex of an ion-exchange resin and phenylpropanol amine with PEG and thus the release of the active substance was slowed down (J. Pharm. Sci. (1981) 70(4), 379-84). Dahl et al. disclose the effects of heat and drying on the release profiles of coated tablets with acetaminophen in comparison with non-pre-treated ones (Drug Dev. Ind. Pharm. (1990) 16 (14) 2097-107). Stamato discloses the effect of the size of particles or emulsion droplets and thus of pores in the second phase of a two-phase coating on the improvement of the release profile (Proc. Int. Symp. Contr. Rel. Bioact. Mater. 19th (1992) 383-4). Wagner et al. disclose the effect of dispersion concentration and of the temperature of the curing of eudragite on the reduction of the release of active substance and on a reproducible and stable release profile (World Meet. Pharm., Biopharm. Pharm. Technol., 1st (1995) 383-4). Garcia-Anton et al. disclose an improvement of the release profile by microencapsulating a hydrophilic or hydrophobic active substance (Sci. Conf. Asian Soc. Cosmet. Sci., 3rd (1997) 93-5). Araujo et al. disclose a stable profile of sustained release of phenylpropanolamine in a concentration of 40-80% from spheronized/extruded grains of the active substance and MCC, the grains being coated with EC (Pharm. Technol. (1999) 23(9) 60, 62, 64, 66, 68, 70). The patent application EP-A-1 020 186 discloses tablets for a sustained release of tramadol with a stable release profile during storage, the tablets contain MCC and are coated by an EC dispersion. The patent application WO 2000/74709 discloses polyester microspheres for the stabilization and improvement of the release profile of encapsulated active substances e.g. insulin. Schmidt et al. disclose a stable release profile at storing for 3 months at 20° C. and a reduced release of an active substance from coated pellets in PEG at 40° C. (Int. J. Pharm. (2001) 216(1-2) 9-16). Maejima et al. disclose the effect of a film coating made of talc and triethyl citrate on the stabilization of the release rate of theophyllin in a concentration of 20% from pellets coated with acrylic polymers (Pharm. Dev. & Technol. (2001) 6(2) 211-21). Wesseling et al. disclose the effects of plasticization times, curing conditions, storage times and core properties on the release of an active substance and the reduction and thus a stable profile of the release of theophylline or chlorphenyramine maleate because of a thermal after-treatment, i.e. the curing of coated pellets (Pharm. Dev. & Technol. (2001) 6(3) 325-31). Chen et al. disclose the effect of the composition and the structure of carriers on the release profile of diazepam from microspheres (Shenyang Yaoke Daxue Xuebao (2001) 18(3), 162-5). The document EP 415522 (Examples 1-4) describes a process wherein an aqueous solvent mixture containing isopropanol, water and acetic acid, is added to the active substance (ondansetron), dried and humidified. The obtained active substance crystals have modified physical properties such as reduced crystal size. The patent application EP-A-454 396 discloses an improvement of tabletting properties if the active substance is pre-blended with citric acid, whereas JP patent application 60-163823 discloses e.g. tablets with clarithromycin and citric acid. However, in the patent and other literature from this field no reference was found to be solving the present problem—i.e. to be dealing with or disclosing a pre-treatment or a humidification of an active substance at preparing a formulation, which would make possible or provide a stable and reproducible release profile of an active substance over the whole shelf life. Nor was found a reference dealing with the properties of an active substance extra requiring a stabilization of the release profile.

### THE INVENTIVE SOLUTION

One object of the invention is a method for a physical pre-treatment of an active substance, by which treatment technologically important physical properties of the active substance are so modified that a formulation prepared therefrom, useful for prevention and/or treatment in medicine, has a more stable release profile of the active substance over the whole shelf life of the medicine than it would be the case with the same composition but without pre-treatment.

Technologically important physical properties of pharmaceutical active substances are e.g. particle size, form and porosity, flow properties (flowability, angle of repose), tapped and bulk densities, hydrophilicity/hydrophobicity, contact angles, solubility and dissolution rates, capacity of plastic/elastic deformation and the like.

Physical methods used in pharmaceutical technology for changing or adapting technologically important properties of active substances are e.g. grinding, sieving, milling, micronizing, trituration, adsorption to carriers of a high active surface, granulation, lyophilization, recrystallization and the like.

Thus, by means of a relatively known method a surprising result was achieved—a more stable and more reproducible release profile of an active substance.

A solvent or a mixture of solvents useful in the present invention is characterized by poor solubility of each active substance therein.

The choice of the active substance suitable for the present invention does not depend so much on the therapeutic class it belongs to or on its chemical structure or skeleton, but more on its properties, especially physical ones.

The parameters of an active substance where a pre-treatment may prove sensible:
if its part in the mass of the whole formulation is over 30%, preferably over 40%;
if it is practically insoluble, i.e. less than about 0.1 g/L, in the solvent used, preferably water,
if, in micronized form, it is difficult to directly tablet or encapsulate it;
if its particles are large (d(0.5)>100 μm, d(0.9)>200 μm), brittle and/or porous and as such change their dissolution over time and therefore need to be micronized. Brittle are those particles that begin to crumble when suspended in water and exposed to ultrasound of the power of 5 W in the volume of 1 L (the power density being 5 W/L). Porous are those particles where the specific pore surface represents more than 20% of the whole specific surface.

An example of an active substance corresponding to the above conditions is clarithromycin, e.g. in controlled release pharmaceutical forms.

When by the technology of direct tabletting there are manufactured tablets with clarithromycin having a particle size over 200 μm, their dissolution noticeably increases at aging.

When tablets with clarithromycin having a particle size over 200 μm are manufactured by aqueous granulation technology, their dissolution noticeably decreases at aging—most probably due to a partial recrystallization of clarithromycin during aqueous granulation and drying. Some tablet ingredients additionally affect the extent of the release slowdown (it has been experimentally demonstrated that e.g. citric acid enhances the slowdown).

It has been surprisingly found that changes in the release rate are minimized if micronized clarithromycin with a particle size from d(0.9) up to about 30 μm at the most is used, which is humidified with a minimum amount of water. Thus recrystallization is kept at the lowest possible level. In the case of micronizing clarithromycin with large particles also the particle porosity and brittleness are reduced. Micronized clarithromycin can either be already the product of a basic synthesis process or it may be micronized later from clarithromycin with large particles.

Changes in release rate still perceived in the stabilized formulation under stress conditions of testing (40° C. and 75% air humidity) are not relevant for the relative bioavailability as confirmed by an in vivo study in healthy volunteers.

In view of above findings, however, an aqueous pre-treatment is necessary for purely technological reasons also when micronized clarithromycin is incorporated into a tablet. Namely, the physical properties of micronized clarithromycin are inadequate for direct tabletting or encapsulating. By a humidifying process followed by drying, these properties are changed into technologically favourable ones (better flowability, compressibility) and the active substance is stabilized. Dried clarithromycin then enters the preparation of a dry mixture for tabletting or encapsulating.

For the pre-treatment either micronized clarithromycin is used or a mixture of clarithromycin and one or more auxiliary substances is prepared, which, under stirring, is humidified with water or with an aqueous solution of one or more auxiliary substances (binders, polymers and/or surfactants). The obtained clarithromycin basis is partially dried, sieved and dried up to a desired humidity grade, e.g. 2.5%. To the dry pre-treated clarithromycin a sieved mixture of the remaining formulation ingredients is added, it is blended and tabletted or encapsulated.

For the pre-treatment of clarithromycin any pharmaceutically acceptable excipient from the basic groups of excipients may be used such as:
fillers, e.g. lactose, microcrystalline cellulose, Ca carbonate, Ca sulfate, glyceryl palmitostearate, mannitol, maltodextrin, various kinds of starch and cellulose, Mg oxide and the like;
disintegrants, e.g. Na or Ca carboxymethylcellulose, SiO₂ (aerosil), crospovidone, cellulose and starch derivatives and the like.

For the pre-treatment of clarithromycin (or of a mixture thereof with the above excipients) by means of humidifying, a poor solvent (e.g. water) or a solution in this solvent of one or more excipients from the following groups may be used:
emulgators, e.g. acacia, carbomer, fatty alcohols, polyoxyethylene alkyl ethers, polyoxyethylene derivatives of castor oil, polyoxyethylene sorbitan esters of fatty acids, polyoxyethylene stearates, sorbitan esters, triethanolamine and the like;
binders, e.g. acacia, alginic acid, carbomer, cellulose derivatives, gelatine, vegetable oils, silicates, polyvinylpyrrolidone and the like;
surfactants, which may be of anionic type e.g. Na lauryl sulfate or Na docusate, of cationic type e.g. benzalkonium chloride or benzethonium chloride, or of non-ionic type e.g. glyceryl monooleate, polyvinyl alcohol, sorbitan esters, polyoxyethylene sorbitan or fatty acid esters and the like;
salts with buffer effect, which are Na and Ca salts of polybasic organic acids, e.g. citric or phosphoric acid and the like.

The pre-treated clarithromycin is the starting material for a direct tabletting or encapsulating mixture, where during the compression process itself a matrix is formed, e.g. a lipid-hydrophilic skeleton controlling the clarithromycin release over 24 hours as e.g. disclosed in SI patent 20150.

Tablets with a high dose of the pre-treated active substance may be very elastic and consequently poorly compressible, so that they have a relatively low hardness. It is usually very difficult to film-coat such tablets.

A further object of the invention is a coating overcoming these difficulties.

Tablet cores with a high dose of the pre-treated active substance may, in their physical properties, differ from tablet cores manufactured according to usual, already known processes.

The changed physical properties of tablet cores required a more rigid film coating, which was achieved in such a way that into a usual film-coating composition (wherein the film-forming agent is a polymer of a lower molecular weight and of a viscosity of about 6 mPas) a polymer of a higher molecular weight and of a viscosity over about 6 mPas, preferably of a viscosity of about 15 mPas, was added. Thereby the effectiveness of the coating of tablet cores with a high dose of a pre-treated active substance was highly improved.

As polymers, cellulose ethers such as e.g. hydroxypropylmethylcellulose and hydroxypropylcellulose can be used.

The mass ratio between the polymers of higher and lower molecular weights in the film coating is at least about 1:9, preferably about 3:7.

Other ingredients in the film coating may be the usual ones, e.g. plasticizers, fillers, colouring agents, polishing agents. As solvents e.g. water or ethanol may be used.

Simultaneously, this film coating also provides for the masking of a possible unpleasant taste of an active substance.

A pharmaceutical formulation prepared from an active substance modified according to the invention may be used in the treatment and prevention of diseases known for each specific substance, e.g. if the active substance is clarithromycin, in the treatment and prevention of bacterial infections.

The invention is illustrated by, but in no way limited to the following Examples.

### EXAMPLE 1

### Composition of a Tablet

Core

| | | |
|---|---|---|
| micronized clarithromycin | 500.0 | mg |
| HPMC E50 Premium ® | 200.0 | mg |
| glyceryl behenate | 250.0 | mg |
| polyvinylpyrrolidone K-25 ® | 60.0 | mg |
| microcrystalline cellulose | 35.5 | mg |
| stearic acid | 15.0 | mg |
| SiO₂ (aerosil 200) | 5.0 | mg |
| Ca stearate | 25.0 | mg |
| talc | 5.0 | mg |
| polyoxyethylene 20 oleate (polysorbate 80V ®) | 24.5 | mg |
| demineralized water | 110.0 | mg |

Clarithromycin and a major part of PVP were pre-treated with an aqueous solution of PVP (minor part) and of polysorbate during stirring in a processor and then dried in a stream of hot air. The dry clarithromycin basis was homogenously blended with the excipients HPMC, glyceryl behenate, microcrystalline cellulose, Ca stearate, stearic acid, aerosil and talc. The mixture was tabletted.

### EXAMPLE 2

As Example 1 with the difference that a dry mixture of clarithromycin and of the whole amount of PVP was prepared and that it was humidified with water.

### EXAMPLE 3

As Example 1 with the difference that a dry mixture of clarithromycin and of the whole amount of PVP was prepared and that it was humidified with an aqueous Na lauryl sulfate solution.

### EXAMPLE 4

As Example 1 with the difference that a dry mixture of clarithromycin and of the whole amount of PVP was prepared and that it was humidified with an aqueous polysorbate 80 solution.

### EXAMPLE 5

A core prepared with compositions or according to processes of Examples 1 to 4 may be coated:

Coating

| | | |
|---|---|---|
| hydroxypropylmethylcellulose (6 mPas) | 14.0 | mg |
| hydroxypropylmethylcellulose (15 mPas) | 6.0 | mg |
| hydroxypropylcellulose | 5.6 | mg |
| polyethylene glycol | 2.0 | mg |
| iron oxide | 0.5 | mg |
| titanium dioxide | 8.1 | mg |
| vanilla aroma | 1.0 | mg |
| talc | 2.8 | mg |
| ethanol | 335.3 | mg |
| demineralized water | 45.7 | mg |
| talc | 0.7 | mg |

From hydroxypropylmethylcelluloses, hydroxypropylcellulose, iron oxide, titanium dioxide, polyethylene glycol, talc and aroma a dispersion in a mixture of ethanol and demineralized water was prepared and tablet cores were coated with this dispersion. Finally, the tablets were polished with talc.

1. A method for a physical pre-treatment of an active substance, characterized in that it comprises adding a poor solvent or a mixture of solvents to the active substance or to a mixture of the active substance with other excipients, the solubility of the substance in said solvent being less than 0.1 g/L, followed by drying.

2. A method for a physical pre-treatment of an active substance according to claim 1, characterized in that said method comprises humidifying with water.

3. A method for a physical pre-treatment of an active substance according to claim 2, characterized in that the aqueous solution may contain various pharmaceutically acceptable excipients such as binders, buffers, emulgators, surfactants and others.

4. A method for a physical pre-treatment of an active substance according to claim 1, characterized in that the part of the active substance in the mass of the whole formulation is over about 30%.

5. A method for a physical pre-treatment of an active substance according to claim 1, characterized in that the part of the active substance in the mass of the whole formulation is over about 40%.

6. A method for a physical pre-treatment of an active substance according to claim 1, characterized in that the active substance is practically insoluble in the solvent used.

7. A method for a physical pre-treatment of an active substance according to claim 6, characterized in that the solvent used is water, wherein the solubility of the active substance is under about 0.1 g/L.

8. A method for a physical pre-treatment of an active substance according to claim 1, characterized in that the active substance, if micronized, is difficult to be directly tabletted or encapsulated.

9. A method for a physical pre-treatment of an active substance according to claim 1, characterized in that the particles thereof are large, brittle and/or porous.

10. A method for a physical pre-treatment of an active substance according to claim 1, characterized in that the active substance is clarithromycin.

11. A method for a physical pre-treatment of an active substance according to claim 10, characterized in that clarithromycin is micronized.

12. A method for a physical pre-treatment of an active substance according to claim 11, characterized in that the pre-treated, micronized clarithromycin enters a direct mixture for tabletting or encapsulating as a starting material.

13. A method for a physical pre-treatment of an active substance according to claim 12, characterized in that the obtained cores are coated.

14. A method for a physical pre-treatment of an active substance according to claim 13, characterized in that the coating also contains a polymer having viscosity of up to about 15 mPas.

15. A method for a physical pre-treatment of an active substance according to claim 14, characterized in that the coating contains at least about 10% of a polymer having viscosity of up to about 15 mPas.

16. A method for a physical pre-treatment of an active substance according to claim 14, characterized in that the polymer used in the coating has a viscosity of over about 6 mPas.

17. A pharmaceutical formulation with clarithromycin or analogues thereof, characterized in that the active substance is modified according to the method of claim 1.

18. A pharmaceutical formulation prepared according to the method of claim 1 for use in medicine for the treatment and prevention of diseases.

19. The use of a film coating composed of a combination of polymers having viscosities of up to about 15 mPas and about 6 mPas for coating tablet cores manufactured according to the method of claim 1.
